(19) United States
(12) Patent Application Publication
(10) Pub. No.: US 2019/0328295 A1
(43) Pub. Date: Oct. 31, 2019

(54) PLANAR ELECTRODES FOR INVASIVE BIOSENSORS

(71) Applicant: Verily Life Sciences LLC, South San Francisco, CA (US)

(72) Inventors: Zenghe Liu, Alameda, CA (US); Todd Whitehurst, Belmont, CA (US); Aurang Zeb, Fremont, CA (US)

(73) Assignee: Verily Life Sciences LLC, South San Francisco, CA (US)

(21) Appl. No.: 16/390,487

(22) Filed: Apr. 22, 2019

Related U.S. Application Data
(60) Provisional application No. 62/663,092, filed on Apr. 26, 2018.

(51) Int. Cl. A61B 5/1486; A61B 5/145

(52) U.S. Cl. CPC A61B 5/14865; A61B 5/14532; A61B 2562/164; A61B 2562/166; A61B 5/14546

(57) ABSTRACT

One example device includes a housing attachable to a wearer's skin; a electrode assembly holder disposed within the housing, the electrode assembly holder comprising: a substrate; a plurality of electrical contacts formed on the substrate; and an electrode assembly electrically coupled to the plurality of electrical contacts, the electrode assembly having a planar surface and comprising: a first screen-printed electrode having first and second ends; a chemical sensing material disposed on the first end of the electrode; and a polymer coating applied to the electrode; and wherein the first end of the screen-printed electrode extends outside of the housing and wherein a second end of each electrode is electrically coupled to one electrical contact of the plurality of electrical contacts.

### CROSS-REFERENCE TO RELATED APPLICATIONS

This application claims priority to U.S. Provisional Patent Application No. 62/663,092, filed Apr. 26, 2018, titled “Planar Electrodes for Invasive Biosensors,” the entirety of which is hereby incorporated by reference.

### FIELD

The present disclosure generally relates to sensor electrodes, and more particularly relates to planar electrodes for invasive biosensors.

### BACKGROUND

Biosensors may employ sensor wires that are inserted into a wearer's skin to detect the presence of an analyte, such as glucose, and provide an electrical signal indicating the amount or concentration of the analyte present. Such sensor wires may have diameters on the order of 100 microns, making them somewhat fragile. Thus, in some cases a wearer may apply a biosensor by first puncturing the wearer's skin with a needle and inserting the sensor wire through the puncture, and then affixing, e.g., adhering, the biosensor to the wearer's skin.

### SUMMARY

Various examples are described for planar electrodes for invasive biosensors. For example, one example device includes a housing attachable to a wearer's skin; an electrode assembly holder disposed within the housing, the electrode assembly holder comprising: a printed circuit board (“PCB”); a plurality of electrical contacts formed on the PCB; and an electrode assembly physically coupled to the PCB, the electrode assembly having a planar surface and having an invasive end and a device end, the electrode assembly comprising: a stack of alternating insulating and electrode layers, each layer screen-printed on a previous layer, and the stack formed on a substrate layer; a first chemical sensing material disposed on an invasive end of a first electrode layer of the electrode layers, a second chemical sensing material disposed on an invasive end of a second electrode layer of the electrode layers, the second chemical sensing material different from the first chemical sensing material; a polymer coating covering at least a portion of the invasive end of the electrode assembly; and wherein: the invasive end of the electrode assembly is insertable beneath the wearer's skin to sense multiple different analyte materials in the wearer's interstitial fluid; a device end of each electrode of the electrode assembly is electrically coupled to one of the electrical contacts; and the invasive end of the electrode assembly extends outside of the housing.

One example method includes providing a substrate material, the substrate material comprising a non-conductive material; screen-printing an electrode on the substrate material using a conductive material, the electrode having first and second ends opposite each other; applying a chemical sensing material to the first end of the electrode; and applying a polymer coating to at least an invasive end of the electrode.

One example electrode assembly includes a screen-printed electrode having first and second ends; a chemical sensing material disposed on the first end of the electrode; and a polymer coating covering at least a portion of an invasive end of the screen-printed electrode and the chemical sensing material.

Another example device includes a housing attachable to a wearer's skin; a electrode assembly holder disposed within the housing, the electrode assembly holder comprising: a substrate; a plurality of electrical contacts formed on the substrate; and an electrode assembly electrically coupled to the plurality of electrical contacts, the electrode assembly having a planar surface and comprising: a first screen-printed electrode having first and second ends; a chemical sensing material disposed on the first end of the electrode; and a polymer coating applied to the electrode; and wherein the first end of the screen-printed electrode extends outside of the housing and wherein a second end of each electrode is electrically coupled to one electrical contact of the plurality of electrical contacts.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIGS. 1A-1B show an example planar electrode for an invasive biosensor;

FIGS. 2A-2F show example planar electrodes for invasive biosensors;

FIGS. 3A-3B show an example planar electrode assembly for an invasive biosensor;

FIG. 4 shows an example planar electrode assembly for an invasive biosensor;

FIGS. 5A-5B show an example planar electrode assembly for an invasive biosensor;

FIG. 6A shows an example electrode assembly holder having an example electrode assembly for an invasive biosensor;

FIGS. 6B-6E show an example wearable biosensor having an example electrode assembly;

FIG. 7 shows an example method for manufacturing a planar electrode assembly for an invasive biosensor; and

FIG. 8 shows an example method for manufacturing planar electrode assemblies for invasive biosensors.

### DETAILED DESCRIPTION

Examples are described herein in the context of planar electrodes for invasive biosensors. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

An example wearable biosensor according to this disclosure, such as a continuous glucose monitor, may include one or more electrodes to be inserted into a wearer's skin or otherwise into the wearer's body, and thus are generally referred to as invasive electrodes. However, rather than the example electrode having a generally cylindrical shape with a substantially circular cross-section (in a plane orthogonal to the length of the electrode), the example biosensor employs a planar electrode.

A planar electrode refers to an electrode that has at least one substantially planar surface running the length of the electrode. Thus, rather than having a substantially circular cross-section, as described above, a cross-section of a planar electrode may be substantially rectangular (or polygonal) or may have one substantially planar surface that adjoins to a curved or other non-planar surface, e.g., in a half-moon cross-section.

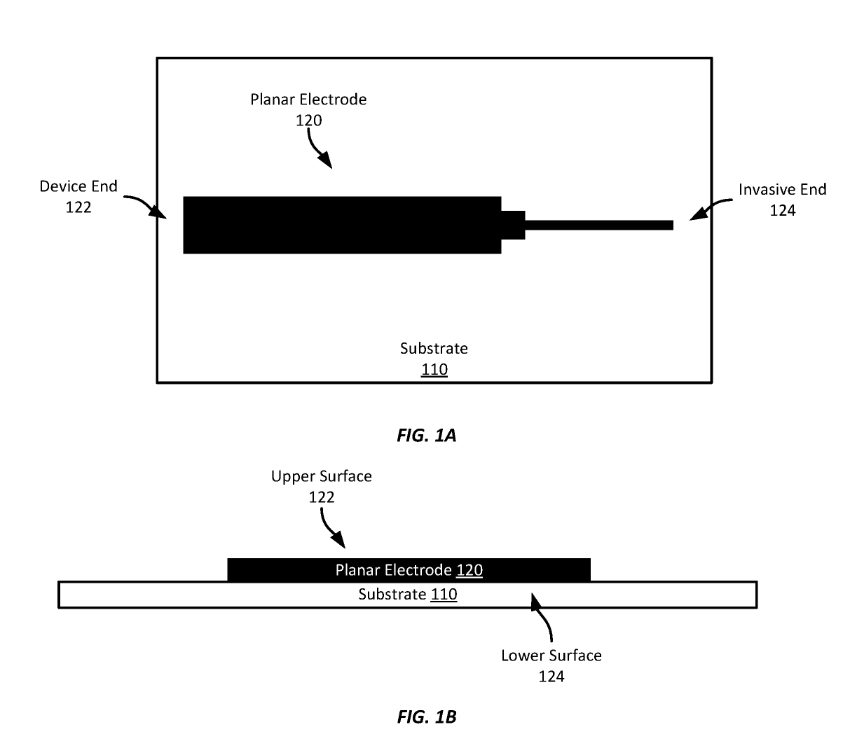

For example, referring to FIGS. 1A-1B, FIG. 1A shows an example planar electrode 100 shown from a top-down view of the upper surface of the electrode 100. The electrode 120 has been screen printed on a substrate 110. The upper surface 122 is substantially planar as is the lower surface 124—the surface opposite the upper surface 122 and in a substantially parallel plane. The surface on the edges of the electrode 120 are substantially perpendicular to the upper and lower surfaces 122, 124 in this example, though in some examples, the sides may be formed to be in planes oblique to the upper or lower surfaces 122, 124. The longitudinal cross-section (the cross-section taken in a plane orthogonal to the longitudinal axis or length of the electrode) is shown in FIG. 1B. As can be seen, the cross-section in this example is substantially rectangular, though it may have any other suitable polygonal shape, such as a trapezoid, parallelogram, etc.

In this example, the planar electrode 120 is formed on a substrate 110 using a screen-printing process. Specifically, the planar electrode 120 is screen printed on the substrate in the shape shown using a platinized carbon ink The perimeter of the planar electrode 120 is designed, in this example, to provide a wider portion at one end 122, where the electrode 120 will be physically or electrically coupled to a sensor device (referred to as the “device end”), and a narrower portion at the other end 124, which will be inserted into the wearer's skin (referred to as the “invasive end”).

In this example, the electrode 120 is the entirety of an invasive sensor wire to be used with an invasive biosensor; however, as will described in more detail below, an invasive sensor wire may include multiple electrodes formed in a stack on top of each other, separated by insulation layers, thereby providing a single electrode assembly that provides multiple discrete electrodes. Such sensor wire assemblies may enable sensing of multiple different analytes with a single sensor wire. Screen printing the electrode may enable manufacturing the electrodes in different shapes or sizes according to different application requirements. In addition, it may facilitate creating a single electrode assembly that includes multiple different electrodes having sensing chemicals.

Further, because the fabrication of the electrode assembly may be performed entirely using screen printing techniques (described below), formation and handling of the sensor wire during the sensor manufacturing process may be easier than provided by conventional techniques. For example, due to the size and shape of the sensor wire assemblies, high-volume manufacturing processes, e.g., automated processes that may include robotic components, may have difficulty picking up, grasping, or otherwise handling the sensor wires without damaging them. However, because some examples according to this disclosure are screen printed on substrate backings, the robot may be able to grasp the substrate and avoid contact with the electrode assembly itself. Thus, in addition to providing a different electrode assembly configuration, the screen printing process may also facilitate the manufacture of the sensor device itself.

In some examples, an electrode assembly with a stack of alternating electrode and insulation layers that have been screen-printed on a planar substrate, where multiple electrode layers have a sensing chemical (in any desirable combination), and where none of the electrode layers provide a working or counter electrode, may provide a desirable analyte sensor implementation. Such an electrode assembly may be easily constructed using screen-printing techniques, may be easily handled and manipulated during the manufacturing process due to the planar substrate material (on which the electrode assembly is formed), and may enable sensing multiple different analytes by the same wearable sensor at substantially the same location within the wearer's skin. Thus, such a monolithic electrode assembly provides advantages in construction of electrode assemblies and manufacture of wearable biosensors.

Further, because a single electrode assembly can sense multiple different analytes via single insertion point into a wearer's skin, other advantages may be realized. For example, one of the electrode layers may not have a sensing chemical and thus may provide a “blank” electrode that provides information about signal interference at the sensing location, which may be usable with respect to each different sensed analyte. Further, because the invasive portion of the electrode assembly is inserted through the same insertion point, the wearer only needs to puncture their skin once and wear a single biosensor, which may reduce discomfort and impact on the wearer when the biosensor is applied. In addition, a monolithic sensor assembly that lacks a counter electrode and a reference electrode may reduce the overall size of the electrode assembly, reduce the complexity of design and manufacture of the assembly, and may enable the use of non-invasive counter or reference electrodes.

In addition, some example electrode assemblies may include one or more working electrodes, but may not include either or both of a counter electrode or a reference electrode. For example, one or both of a counter or reference electrodes may be provided via a non-invasive electrode(s), such as an electrode attached to the wearer's skin. One advantage of some such arrangements may be that the electrode assembly may be substantially planar and cause less discomfort to the wearer upon insertion or while it is worn. Further, in examples where the electrode assembly lacks either or both of a counter or reference electrode, the electrode assembly may be thin enough to stack multiple working electrodes in the same electrode assembly, rather than devoting one or more layers to a working or counter electrode. Still further advantages according to this disclosure may be realized according to different examples.

This illustrative example is given to introduce the reader to the general subject matter discussed herein and the disclosure is not limited to this example. The following sections describe various additional non-limiting examples and examples of planar electrodes for invasive biosensors.

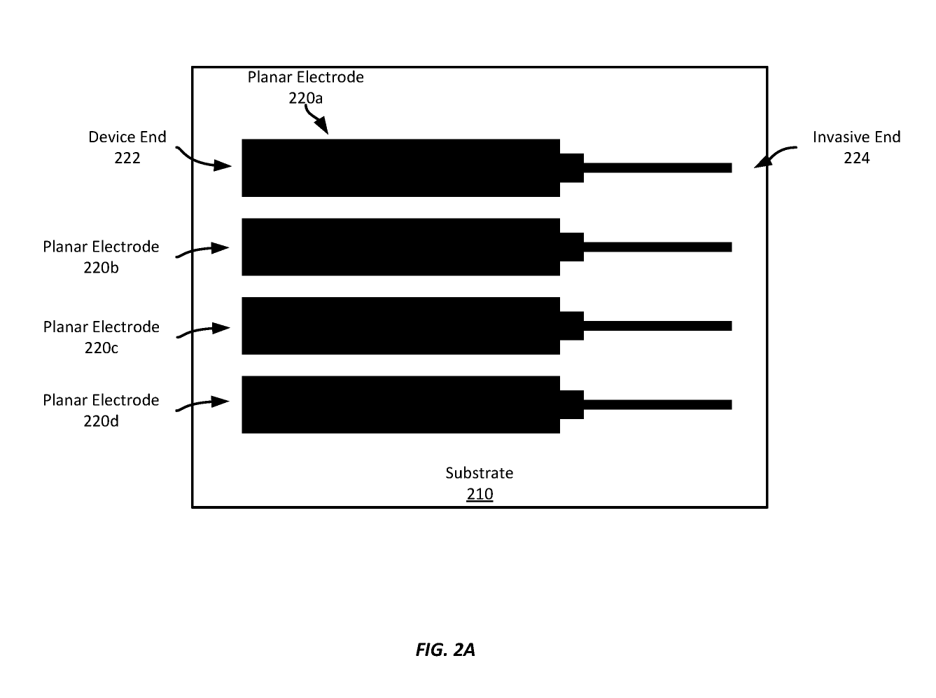

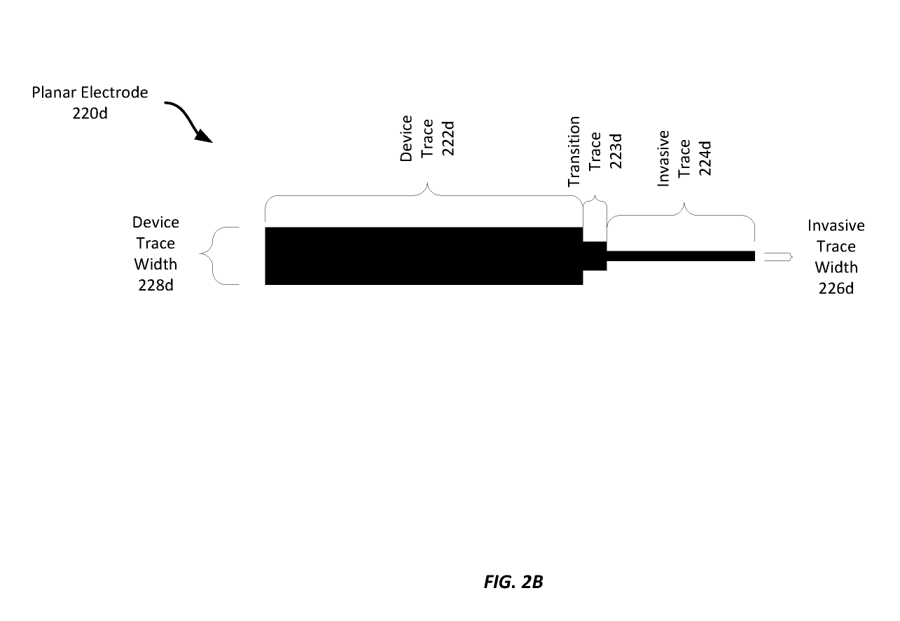

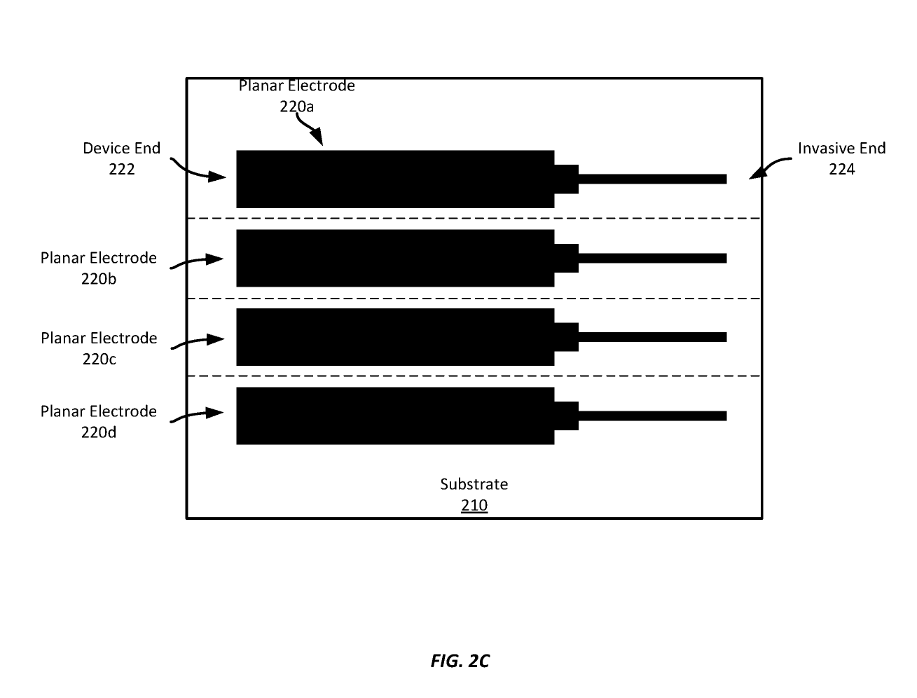

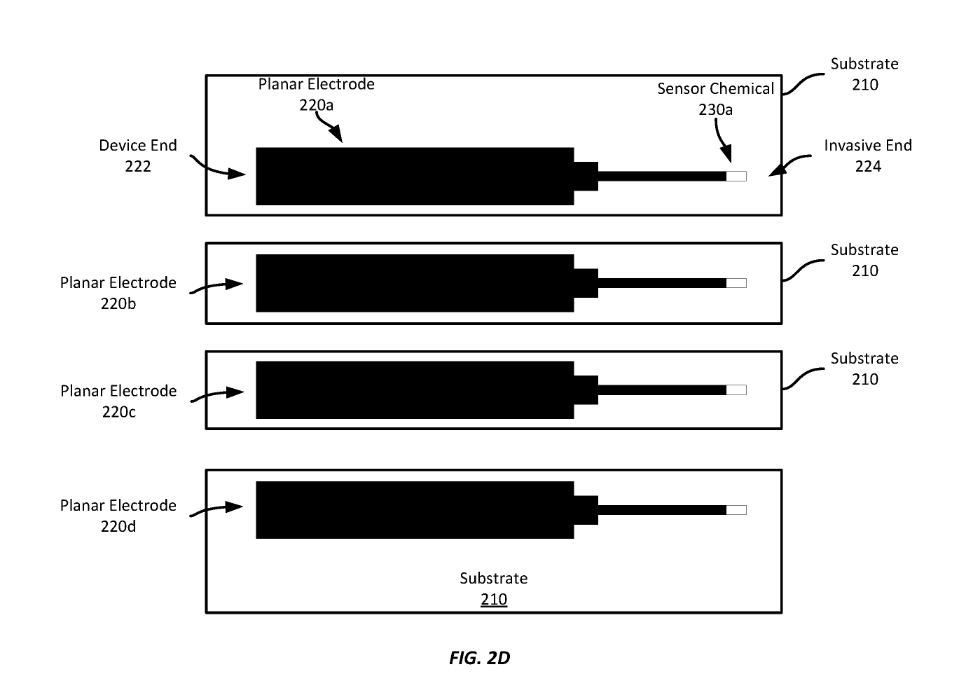

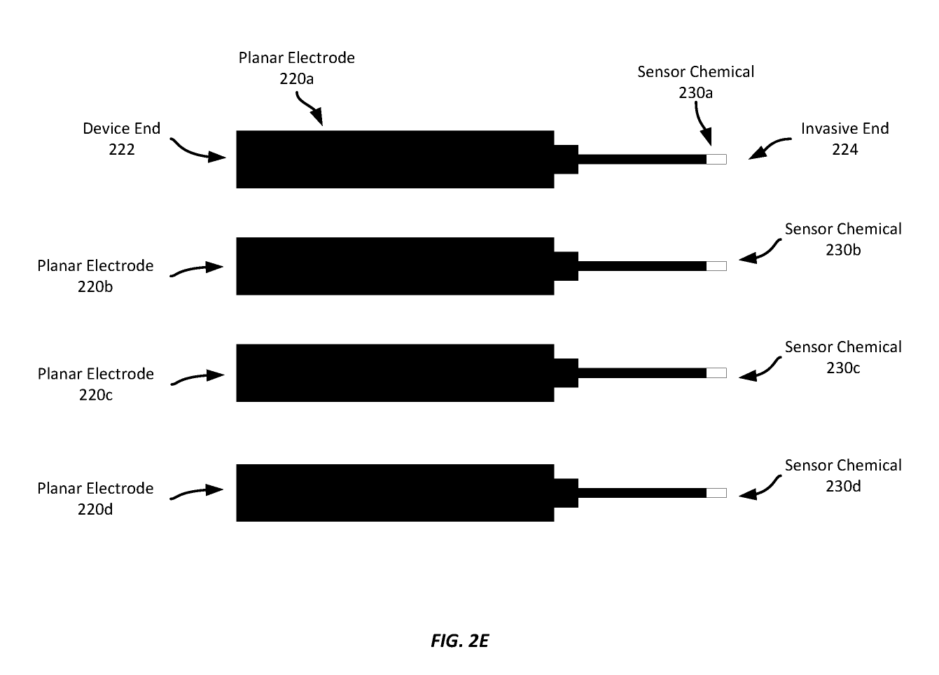

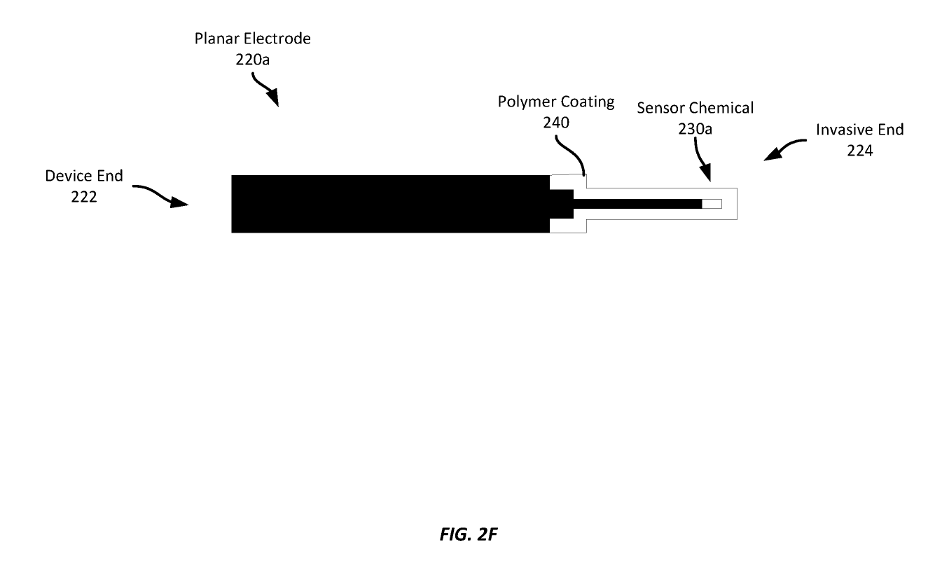

Referring now to FIGS. 2A-2F, FIGS. 2A-2F show a progression of forming planar electrodes on a common substrate, example dimensions of planar electrodes, singulating the discrete electrodes, separating the electrodes from the substrate, applying sensing chemistry to the invasive ends of the electrodes, and applying a polymer coating to the electrodes.

Beginning with FIG. 2A, a sheet of substrate material 210 is provided to a screen-printing device, which screen prints multiple planar electrodes 220a-d on the substrate material. Any suitable substrate may be employed, such as polyethylene terephthalate (“PET”), polyimide, or any other suitable plastic or otherwise non-conductive material. Any suitable substrate thickness may be used. For example, a substrate layer of at least 5 mils, where 1 mil is 0.001 inches, may be employed in some examples. Other examples may employ thinner or thicker substrate layers, such as between 3 to 10 mils.

One or more planar electrodes 220a-d may then be screen printed on the substrate material 210, separated from adjacent electrodes by a suitable gap, such as 1-2 millimeters (“mm”) or more. In some examples, the distance between discrete electrodes may be set based on a midline of each electrode. For example, electrodes may be formed such that the midline of one electrode is spaced 10 mm from the midline of each adjacent electrode.

The electrodes 220a-d may be screen printed using any suitable conductive material. For example, platinized carbon ink, a conductive carbon ink, or any other suitable conductive ink may be employed according to some examples. The electrodes 220a-d may be screen printed with a predetermined thickness, such as 5 microns or greater.

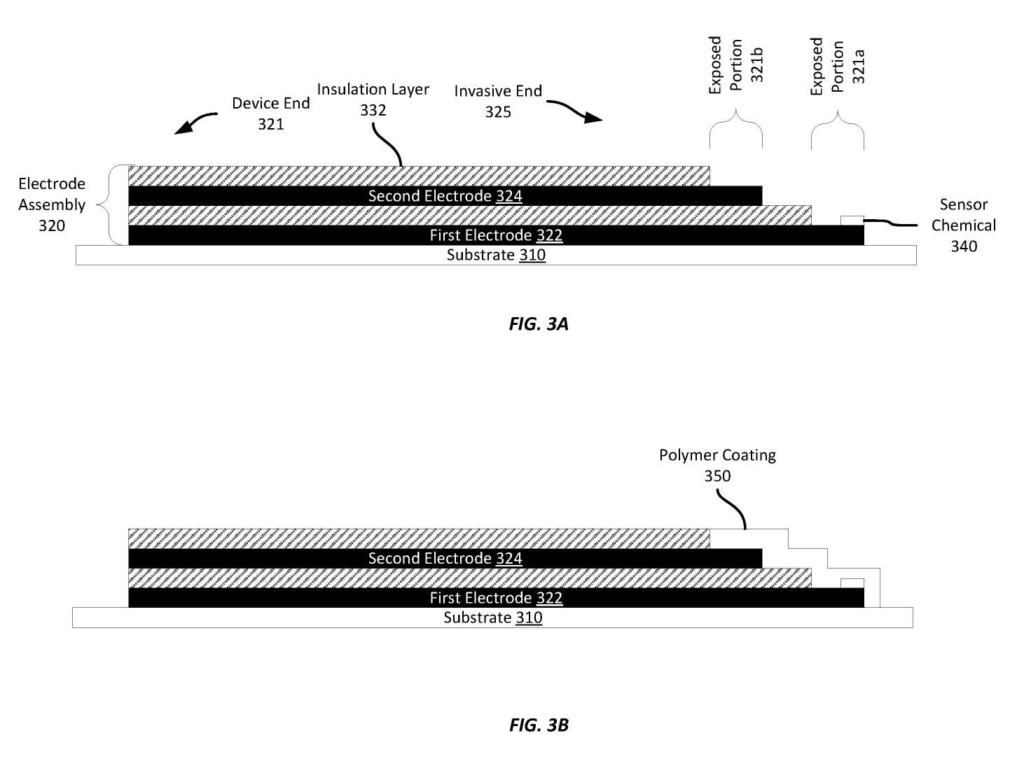

In this example, the planar electrodes 220a-d each include an electrode assembly having only a single electrode having a thickness of between substantially 1 to 15 microns. However, as will be discussed in more detail below with respect to FIGS. 3 to 6, the planar electrode 220a-d may be part of an electrode assembly having multiple electrodes formed in a stack, interleaved with nonconductive layers. Thus, the example shown in FIGS. 2A-2F are equally applicable to such electrode assemblies.

FIG. 2B illustrates the shape and dimensions of one example planar electrode 220d. In this example, the planar electrode 220d includes a device trace portion 222d, a transition trace portion 223d, and an invasive trace portion 224d. In this example, the device trace portion 222d has a length of substantially 5 mm and a width 228d of substantially 1 mm, the invasive trace portion 224d has a length of substantially 1 mm and a width 226d of substantially 0.1 mm. However, any suitable dimensions may be employed. For example, the device trace portion 222d may have any suitable length, such as between 5 mm to 20 mm, and width, such as between 1 mm to 5 mm. The invasive trace portion 224d may have any suitable length of between 0.5 mm to 20 mm and a width 226d between 0.1 mm to 0.5 mm However, still other suitable dimensions may be used based on the requirements of a particular application. In addition to the device trace 222d and the invasive trace 224d, the planar electrode 220d also includes a transition trace 223d that connects the device trace 222d to the invasive trace 224d. Such a transition trace 223d may be any suitable length or width or may be omitted entirely in some examples.

Referring now to FIG. 2C, FIG. 2C illustrates the planar electrodes 220a-d of FIG. 2A formed on the substrate material 210, but illustrates where singulation cuts will be made in the substrate material to singulate the discrete electrodes. “Singulation” refers to the process of cutting the substrate material 210 to separate electrodes from other electrodes so that they may be handled separately, rather than as a group on a common sheet of substrate material. Singulation may be performed at any suitable point within the manufacturing process. Further, it should be appreciated that “singulation” does not require that each electrode be separated from every other electrode. In some examples, singulation may separate electrodes into pairs (or triplets, etc.), where each pair of electrodes may be installed in the same biosensor. Singulation may enable easier handling of the planar electrodes during the manufacturing process of the biosensors, such as by enabling a picking machine to pick the singulated electrodes for transfer to another station where the substrate material is removed and the electrode assembly is installed in a biosensor.

In FIG. 2D, the singulated planar electrodes 220a-d have each had a sensor chemical 230a-d (230b-d not labelled, but applied to electrodes 220b-d, respectively) applied to the invasive end of the respective electrode 220a-d. The sensor chemicals 230a-d may be any suitable sensor chemical, such as glucose oxidase (“GOX”), any suitable alcohol oxidase, a cholesterol oxidase, lactate oxidase, etc. A predetermined quantity of such sensor chemicals (also referred to as “sensor chemistry”) is applied to the invasive end 224 of each electrode shown in FIG. 2D. It should be appreciated that sensor chemistry may or may not be applied to any individual electrode, depending on its design. For example, an electrode may be a blank electrode to correct electrochemical interferences or provide warnings to wearers if the interference signal shows up on this electrode Further, and as will be described in greater detail below, an electrode or sensor assembly may include multiple electrodes, each of which may have a sensor chemical applied to the invasive end, where the sensor chemicals may be different for each electrode in the stack, or one or more electrodes within a stack may not have a sensor chemical applied to it.

FIG. 2E illustrates each of the planar electrodes 220a-d after being removed from the substrate material 210 (not shown in FIG. 2E). The planar electrodes 220a-d may now be installed into a biosensor assembly. In some examples, a further layer may be applied to the planar electrodes 220a-d before it is installed into a biosensor assembly. For example, an additional protective layer of a polymer coating may be applied, such as by dipping the invasive end of the electrode(s) into a liquid polymer bath. Such a protective coating may be any suitable polymer material, such as a polyurethane material.

For example, FIG. 2F illustrates one of the planar electrodes 220a after having a polymer coating 240 applied to it. In this example, the planar electrode 220a was dipped into a liquid polymer bath, which applies a layer of the polymer material 240 over the invasive end 325 of the electrode and sensor chemical 230a, which may provide protection against physical damage to the electrode or sensor chemical 230a, and provide biocompatibility during the wearing of the sensor by a wearer.

Referring now to FIGS. 3A-3B, FIG. 3A shows a side cross-section of an example electrode assembly 320 formed on a substrate material 310. The electrode assembly 320 in this example has two electrode layers, identified as the first electrode 322 and the second electrode 324 in FIG. 3A. Each electrode 322-324 is screen printed and they are insulated from each other by an intervening insulation layer 330. The electrodes 322, 324 in this example are formed by screen printing the first electrode 322 on the substrate 310, then screen printing the insulation layer 330 on top of the first electrode 322, and then screen printing the second electrode 324 on top of the insulation layer 330. The electrodes 322, 324 may be formed of any suitable conductive material, such as the platinized carbon ink, carbon ink, etc. discussed above. The insulation layer 330 may be any suitable material, including any suitable printable dielectric ink.

As can be seen in FIG. 3A, insulation layers 330, 332 are formed to be shorter in length than the prior electrode layer, thereby exposing a portion of the prior electrode layer to enable it to have a sensor chemical 340 deposited on an invasive end 325, opposite the device end 321, and to allow the electrode to be exposed to interstitial fluid when inserted into a wearer's skin. The amount of an electrode layer to remain exposed may be selected based on the amount of sensor chemistry to be applied or an amount of the electrode surface to be exposed to the interstitial fluid. For example, the exposed portion 321 may be between substantially 0.25 to 1.0 mm.

The thickness (shown in FIG. 3A) of each electrode or insulation layer may be established based on the application. For example, each electrode and insulation layer may have a thickness of substantially 5 microns; however, any suitable thickness may be employed. Further, while the example shown in FIG. 3 includes two electrodes 322, 324 separated by an insulation layer 330 (with a further insulation layer 332 screen-printed on the second electrode 324), any suitable number of alternating electrode and insulation layers may be formed according to different examples. Further, as discussed above, a polymer coating 350, such as the one shown in FIG. 3B, may be applied to the invasive end 325 of the electrode assembly 320 after all electrode and insulation layers have been formed, either before or after singulation.

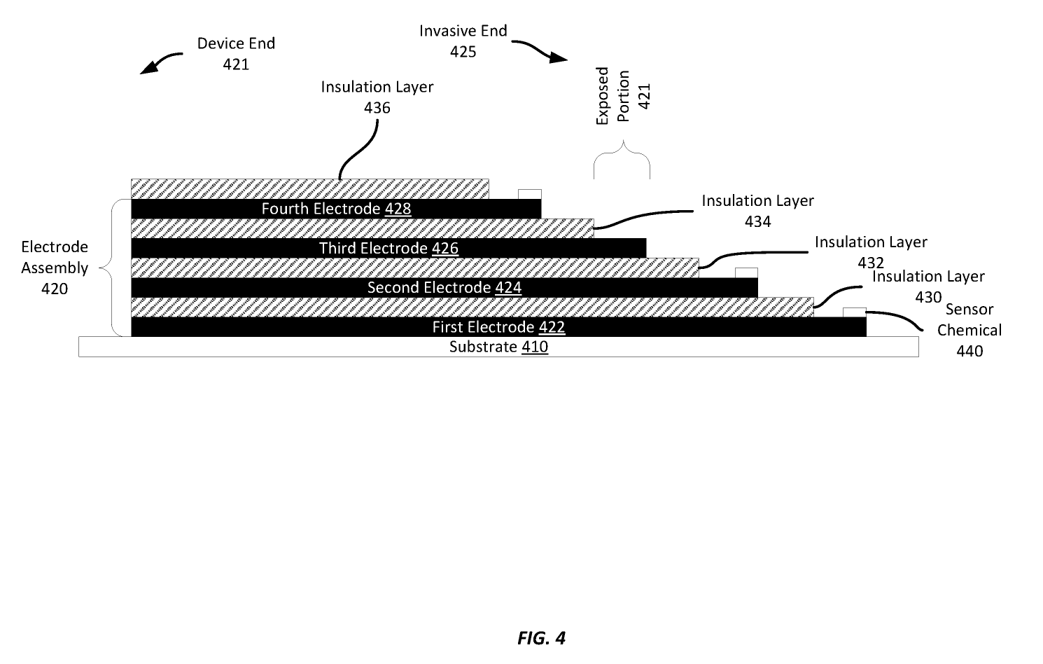

For example, referring to FIG. 4, FIG. 4 shows an example electrode assembly 420 having four electrodes 422-428 formed in a stack, with intervening insulation layers 430-434 formed between successive electrodes 422-428, and an insulation layer 436 formed on the fourth electrode 428. As with the example shown in FIG. 3, each electrode layer 422-428 has an exposed portion 421 that may have a sensor chemical 440 deposited on it. The exposed portions 421 are configured to be exposed to interstitial fluid when inserted into the wearer's skin. The applied sensor chemicals 440 may each be different per electrode layer, or the same sensor chemical may be applied to two or more layers, or any combination of the same or different sensor chemicals may be used. Further, and as may be seen in FIG. 4, one or more layers may not have a sensor chemical 440 applied to it. This electrode can be used as a blank electrode to correct electrochemical interferences or provide warnings to wearers if the interference signal shows up on this electrode. Further, and as discussed above, a polymer coating may be applied to the electrode assembly 420.

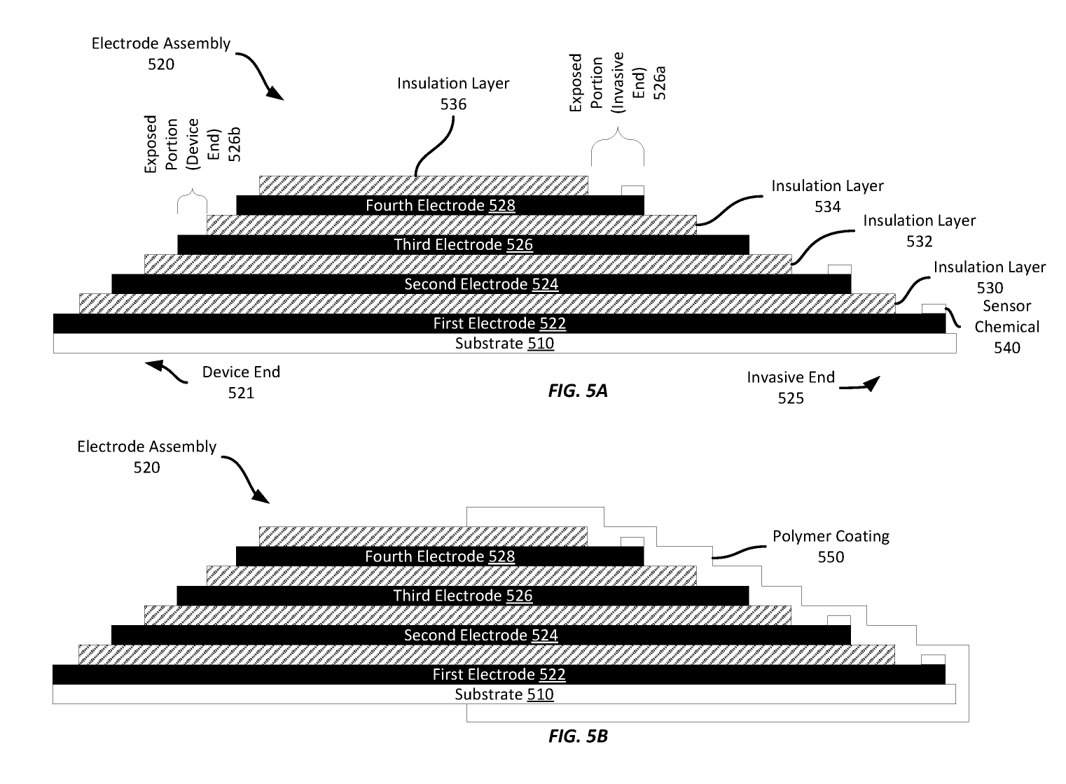

Referring now to FIGS. 5A-5B, FIG. 5A shows another example electrode assembly 520. Like the electrode assembly 420 shown in FIG. 4, the electrode assembly 520 of FIG. 5 includes four electrode layers 522-528 with the invasive end of each electrode 522-528 exposed to provide a region, e.g., 526a, to which a sensor chemical 540 may be applied or to otherwise expose the electrodes to interstitial fluid when inserted into a wearer's skin. Insulation layers 530-534 are screen printed between the electrode layers 522-528 to electrically insulate each electrode from the others. Further, an insulation layer 536 formed on the fourth electrode 528 as shown.

In addition, a portion of the device end of each electrode is also exposed, e.g., 526b. Such a configuration may enable a physical or electrical connection between each electrode 522-528 and a sensor printed circuit board (“PCB”) or other mounting structure. Thus, to the device end of each exposed electrode portion, a coupling device, such as a metal clip or crimp, may be used to physically grip the electrode and to provide an electrical connection to other electronics within the sensor device. In some examples, portions of one or more insulation layers may be exposed to provide additional or alternate locations to physically couple the electrode assembly to a sensor device without a corresponding electrical coupling. In one such example, electrical couplings may be applied to each exposed device portion of each electrode 522-528, and a physical coupling may be applied to each exposed device portion of each insulation layer 530-534.

After the electrode assembly 520 has been screen printed and sensor chemicals 540 have been applied to one or more of the electrodes 522-528, the electrode assembly 520 may have a polymer coating 550 applied to it, such as by dipping the invasive end 525 of the electrode assembly 520 into a bath of a suitable liquid polymer solution. FIG. 5B shows the example electrode assembly 520 of FIG. 5A after polymer coating 550 has been applied to the invasive end 525. Such a polymer coating 550 may provide protection against contamination or damage before the electrode assembly 520 is installed in a biosensor and inserted into a wearer's skin, or it may help provide biocompatibility with the invasive end of the sensor assembly 520.

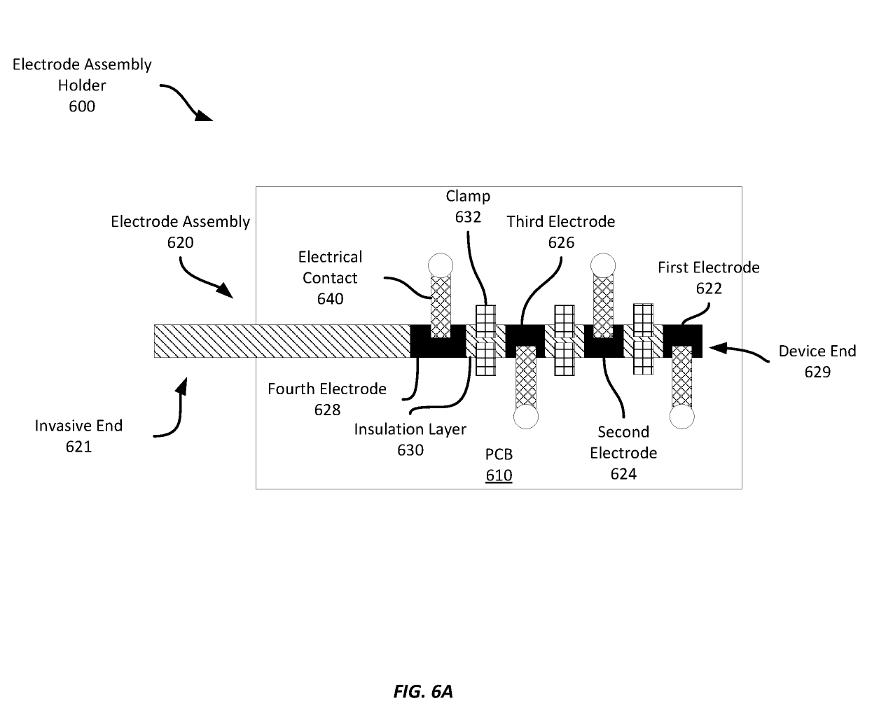

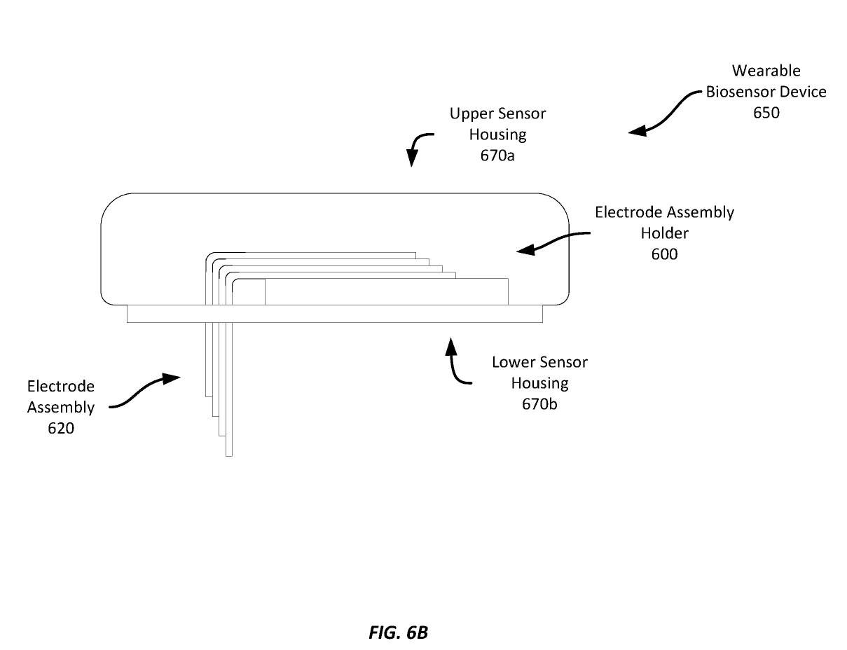

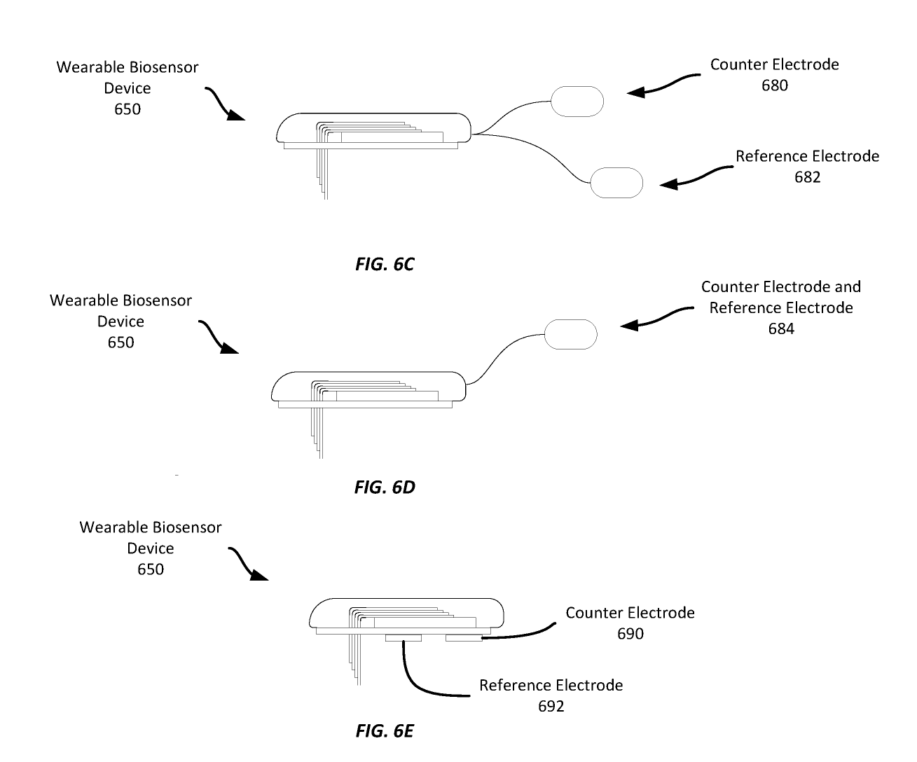

Referring now to FIGS. 6A-6E, FIG. 6A shows a top-down view of a electrode assembly holder 600 having an electrode assembly 620. In this example, the electrode assembly 620 has four electrodes 622-628 assembled in a stack with intervening insulation layers, such as shown in FIGS. 5A-5B and described above. The device end 629 of the electrode assembly 620 has been physically and electrically coupled to a mounting surface, which is a PCB 610 in this example. The invasive end 621 of the electrode assembly 620 extends beyond the edge of the PCB 610 and bends downward (in the direction of the PCB 610) at an angle, e.g., between 30 and 90 degrees, to enable it to be inserted into a wearer's skin.

The electrode assembly 620 has been physically coupled to the PCB by three clamps 632. In this example, the clamps 632 have two halves that are each physically coupled to the PCB 610 and have been bent closed over the exposed portions of the insulation layers 630 of the sensor assembly 620. And while this example employs clamps, other examples may use different means for physically coupling the electrode assembly 620 to the PCB 610, including clasps, leaf springs, etc.

The electrode assembly 620 is also electrically coupled to the PCB 610 via four electrical contacts 640. Each electrode 622-628 is electrically coupled to a different one of the electrical contacts 640 to provide individual electrical connections between the respective electrode and the PCB 610. Each electrical contact 640 in this example runs from the respective electrode 622-628 to a corresponding via, which provides an electrical coupling to other electronics within a suitable sensor device. In this example, each electrical contact 640 includes a leaf spring is coupled to the PCB 610 and presses against the respective electrode 622-628 to provide an electrical coupling. However, it should be understood that any suitable means for electrically coupling an electrode 622-628 may be employed according to different examples, including clamps, crimps, clasps, solder, etc. Further, it should be appreciated that the means for electrically coupling may also provide a physically coupling. Similarly, one or more of the means for physically coupling may be also provide an electrical coupling. For example, the clamps 632 in some examples may be omitted and the electrical contact 640 may provide both a physical and an electrical coupling of the electrode assembly 620 to the PCB 610. In one such example, the electrode assembly may be not provide exposed portions of insulation layers, and instead, may only expose the different electrodes to provide for both physical and electrical coupling, such as shown in FIGS. 5A-5B.

While the example shown in FIG. 6A illustrates the electrode assembly partially disposed within the housing and partially extending outside of the housing, in some examples, the electrode assembly may initially be entirely disposed within the housing. For example, the electrode assembly 620 may be initially configured in a single plane without the bend shown in FIG. 6A. At a later time when the wearable biosensor device 650 is to be affixed to a wearer, the wearer may employ an applicator device that punctures the wearer's skin to allow the electrode assembly to be inserted partially into the wearer's skin. The applicator may also bend the electrode assembly 620 through the lower sensor housing 670b into the configuration shown, or having a bend angle of less than 90 degrees. Such an arrangement may help avoid damage to the electrode assembly 620 while the biosensor 650 is in storage or being handled. Further, in some examples, the electrode assembly 620 may not be installed in the electrode assembly holder 600 until the biosensor 650 is to be used. Rather, the electrode assembly 620 may be provided as a separate component that is installed into the electrode assembly holder 600 and electrically coupled to the electrical contacts 640 on the electrode assembly holder 600 prior to use.

Referring now to FIG. 6B, FIG. 6B shows a side view of an example wearable biosensor device 650 having a two-part sensor housing 670a-b in which the electrode assembly holder 600 is installed. As can be seen in FIG. 6B, the electrode assembly 620 extends downward through the lower sensor housing portion 670b, which is configured to be physically coupled to a wearer's skin; it should be noted that, for ease of depiction, only two of the four electrode layers are depicted in the electrode assembly 620. Application of the wearable biosensor device may involve using a needle to puncture the wearer's skin, inserting the electrode assembly 620 into the puncture, and mounting the wearable biosensor device 650 to the wearer's skin. Thus, once the wearable sensor 650 has been applied to the wearer's skin, the invasive end of the electrode assembly 620 may be held in place in the interstitial fluid beneath the wearer's skin.

FIGS. 6C-6E show example wearable biosensor devices 650 that also include non-invasive conductive pads that may provide either or both of a counter electrode or reference electrode. In these examples, the conductive pads are configured to be attached to the wearer's skin and to provide counter or reference electrode signals to the wearable biosensor device 650. Further, in these examples, the electrode assembly 620 of the wearable biosensor device 650 does not include either a reference electrode or a counter electrode. Instead, such electrodes are provided in non-invasive conductive pads. One advantage of some such arrangements may be that the electrode assembly may be substantially planar and cause less discomfort to the wearer upon insertion or while it is worn. Further, in examples where the electrode assembly lacks either or both of a counter or reference electrode, the electrode assembly may be thin enough to stack multiple working electrodes in the same electrode assembly, rather than devoting one or more layers to a working or counter electrode.

In the example shown in FIG. 6C, the counter electrode 680 and the reference electrode 682 are each a discrete non-invasive conductive pad separate from the wearable biosensor and electrically connected to the wearable biosensor device 650 via a respective wire. Thus, the wearable biosensor device 650 may be affixed to one location on the wearer's skin, while the counter and reference electrodes 680-682 may be affixed at other locations on the wearer's skin. It should be appreciated that while this example includes both a counter electrode 680 and a reference electrode 682, some examples may include only include one of a non-invasive counter electrode 680 or a reference electrode 682.

The example shown in FIG. 6D includes a single non-invasive conductive pad 684 that provides both counter and reference electrode signals. In contrast, the example shown in FIG. 6E includes discrete non-invasive conductive pads providing a counter electrode 690 and a reference electrode 692. These non-invasive conductive pads are coupled to a bottom surface of the wearable biosensor device 650 such that when the wearable biosensor device 650 is affixed to a wearer's skin, the counter electrode 690 and the reference electrode 692 are coupled to the wearer's skin.

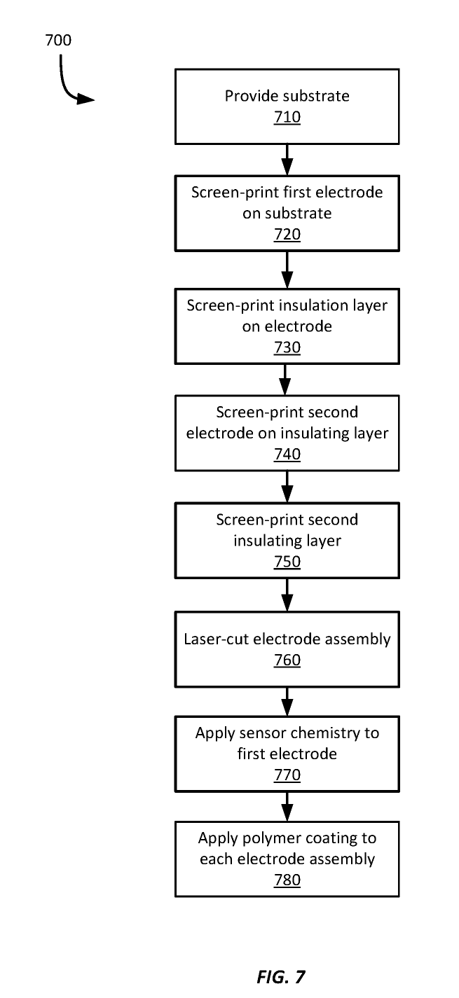

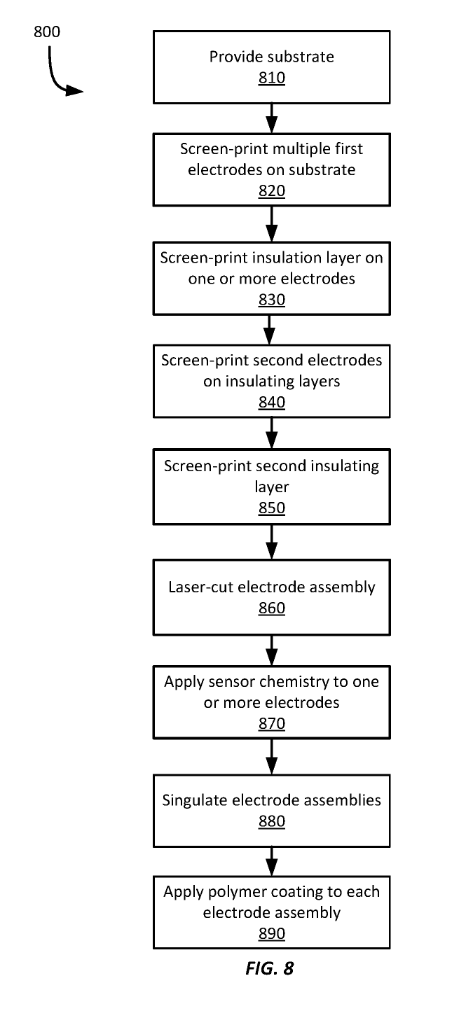

Referring now to FIG. 7, FIG. 7 shows an example method 700 of manufacturing an electrode assembly according to this disclosure. The method 700 of FIG. 7 will be described with respect to the example electrode assembly of FIGS. 3A-3B; however it should be appreciated that such a method may be employed to manufacture any electrode assembly according to this disclosure.

At block 710, substrate material 310 is provided. Suitable substrate materials, such as those described above, include PET, polyimide, or any other suitable plastic or otherwise non-conductive material.

At block 720, a first electrode 322 is screen printed on the substrate material 310. As discussed above, any suitable conductive ink may be employed to screen print the electrode 322, including a platinized carbon ink, a conductive carbon ink, etc. The first electrode 322 may be screen printed in any suitable shape. For example, referring to FIG. 2B, the first electrode may have a device trace 222d and an invasive trace 224d, where the invasive trace width 226d is less than the device trace width 228d. Further, in some examples, the invasive trace 224d may be coupled to the device trace 222d by a transition trace 223d having a width greater than the invasive trace width 226d, but less than the device trace width 228d, as shown in FIG. 2B. However, in some examples, the first electrode may lack a transition trace.

The first electrode 322 may be screen printed with any suitable thickness. A thickness of the first electrode 322 may be established by screen printing the first electrode in layers such that the first electrode has multiple layers of conductive ink to form the first electrode. In some examples the first electrode may be screen printed in a single layer of a predetermined thickness. Suitable thicknesses may range from 1 to 15 microns or more.

At block 730, an insulation layer 330 is screen printed on top of the first electrode 322. In this example, the insulation layer 330 is screen printed using a suitable non-conductive ink, such as a dielectric ink The insulation layer 330 is screen printed to leave a portion of the invasive end of the first electrode 322 exposed without an insulation layer 330 screen printed on it. Such a configuration may allow the first electrode 322 to be exposed to interstitial fluid when inserted into a wearer's skin and to allow a sensor chemical 340 to later be deposited on it. In some examples, a portion of the device end of the first electrode 322 may remain exposed as well, such as may be seen in the example electrode assembly shown in FIGS. 5A-5B.

The thickness of the insulation layer 300 may be established as discussed above with respect to the first electrode, such as by screen printing multiple layers of non-conductive ink or be screen printing a single layer of a suitable thickness. As with the first electrode, suitable thicknesses may range from 1 to 15 microns or more.

At block 740, a second electrode 324 is screen printed on top of the insulation layer 330. The second electrode 324 is screen printed using any suitable conductive ink, such as those discussed above. The second electrode 324 in this example is only screen printed on the insulation layer 330 such that the second electrode 324 is electrically isolated from the first electrode 322. While in some examples it may be desirable to provide an electrical coupling between the first and second electrodes 322, 324, in this example, the two electrodes 322, 324 are electrically isolated by the insulation layer 330. Thus, the second electrode 324 is screen printed on the insulation layer 330. In this example, the second electrode 324 is screen printed such that it is coextensive with the insulation layer 330; however, in some examples, a portion of the insulation layer, at either or both ends of the electrode assembly 320, may remain exposed. An example of one such configuration is shown in FIGS. 5A-5B.

While in this example, only two electrodes 322, 324 are formed, in some examples, more than two electrodes may be formed in a stack by continuing to screen print alternating layers of insulation and electrode material. For example, the example electrode shown in FIGS. 5A-5B may be screen printed by repeating blocks 730-740 to form four electrode layers 522-528 with intervening insulation layers 530-534. Further, in some examples, an electrode assembly may only include a single electrode. Thus, blocks 730 and 740 may not be performed. Further, blocks 730 and 740 may be repeated as many times as needed to create a stack of electrodes and intervening insulation layers according to a particular application.

At block 750, a second insulation layer 332 is screen printed on top of the second electrode 324, generally as discussed above with respect to block 730. After block 750 has been completed, the method 700 may proceed to block 760, or it may return to block 740 to add additional layers of alternating electrodes and insulation layers. Blocks 740-750 may be repeated as many times as needed to create a suitable stack of alternating electrode and insulation layers.

At block 760, the electrode assembly is laser cut to the precise designed dimensions for the electrode assembly. Laser cutting may enable the dimensions of each electrode 322-324 and each insulation layer 330 to be precisely sized and shaped for a particular application. It should be appreciated, however, the laser cutting is optional and the size and shape may be established by the screen printing process.

At block 770, a sensor chemical 340 may be applied to one or more electrodes 322-324 of the electrode assembly 320. Any suitable sensor chemical, such as those described above, may be applied at block 760. In this example, a sensor chemical 340 is applied to the exposed invasive end of the first electrode 322, while no sensor chemical is applied to the second electrode 324. However, sensor chemicals may be applied to any or all electrodes within an electrode assembly. Further, different sensor chemicals may be applied to different electrodes. Such configurations may enable testing of multiple analytes with a single sensor assembly. Alternatively, the same sensor chemical may be applied to multiple electrodes, which may provide multiple sensor signals to sensor electronics for the same analyte, which may provide a more reliable measure of the analyte in the wearer's interstitial fluid. Further, one or more electrodes may not have a sensor chemical applied. In some examples, such electrodes without an applied sensor chemical may provide a reference electrode or another baseline signal to sensor electronics to enable more accurate analyte sensor measurements.

At block 780, a polymer coating 350 is applied to the electrode assembly 320. In this example, the electrode assembly is dipped in a liquid polymer bath. In some examples, however, a liquid polymer may be sprayed onto the electrode assembly 320. For example, the substrate may be used to grasp and dip the electrode assembly into a liquid polymer bath.

After the polymer coating 350 is applied, the electrode assembly 320 may be installed in a sensor device. For example, the electrode assembly 320 may be physically coupled to a PCB or other substrate and electrically coupled to one or more electrical contacts on the PCB or substrate to electrically couple the electrode assembly to sensor electronics within the sensor device.

It should be appreciated that one or more blocks of the method 700 is optional. For example, blocks 730 to 750 may be omitted in examples where an electrode assembly includes only one electrode. Further block 760 may be omitted if laser cutting is not desired or otherwise is not available. Block 780 may be omitted if a polymer coating is not desirable or available. Further, the orderings of the blocks shown in FIG. 7 may be changed according to various examples according to particular implementations. For example, block 770 may be performed before block 760.

Referring now to FIG. 8, FIG. 8 shows an example method 800 of manufacturing multiple electrode assemblies on a single sheet of substrate material according to this disclosure. The method 800 of FIG. 8 will be described with respect to the example electrode assembly of FIGS. 2A-2F and with reference to FIGS. 3A-3B; however it should be appreciated that such a method may be employed to manufacture any electrode assembly according to this disclosure.

At block 810, a sheet of substrate material 210 is provided substantially as described above with respect to block 710.

At block 820, multiple first electrodes 220a-d are screen printed on the substrate material 210 substantially as described above with respect to block 720. Each first electrode is screen printed at a pre-defined spacing on the substrate material 210. The pre-defined spacing may enable the electrodes 220a-d to later be singulated without damaging or cutting the electrodes 220a-d themselves.

At block 830, insulation layers are screen printed on each first electrode 220a-d substantially as discussed above with respect to block 730. In this example, an insulation layer is screen printed on each first electrode; however, depending on the configuration of each electrode assembly to be manufactured, an insulation layer may not be screen printed on one or more first electrodes.

At block 840, a second electrode is screen printed on one or more of the electrode assemblies substantially as described above with respect to block 740. As discussed above, the second electrodes are screen printed on the respective insulation layers. Thus, for any first electrodes that did not have an insulation layer applied, a second electrode may not be screen printed. Further, and as described above, blocks 730-740 may be repeated as many times as desired to create an electrode assembly having any suitable number of electrodes with intervening insulation layers. Since each electrode assembly formed on the substrate material 210 may have a different design, adjacent electrode assemblies may have different numbers of electrodes.

At block 850, a second insulation layer is screen printed on top of the second electrode, generally as discussed above with respect to block 830. After block 850 has been completed, the method 800 may proceed to block 860, or it may return to block 840 to add additional alternating electrode and insulation layers. Blocks 840-850 may be repeated as many times as needed to create a suitable stack of alternating electrode and insulation layers.

At block 860, one or more of the electrode assemblies is laser cut substantially as described above with respect to block 750. Further, and as described above, laser cutting is optional and may not be performed in some examples.

At block 870, a sensor chemical is applied to one or more electrodes of each electrode assembly substantially as described above with respect to block 760. Further, because each discrete sensor assembly may be designed for a different purpose, each sensor assembly may have a different sensor chemical (or multiple sensor chemicals) applied to it according to different examples.

At block 880, the electrode assemblies are singulated as shown in FIG. 2D. Singulation may be performed by laser cutting the substrate materials 210, or by using a mechanical cutting tool, such as a knife, blade, scissors, etc. Further, any other suitable techniques to singulate the electrode assemblies may be employed according to various examples.

At block 890, a polymer coating is applied to each electrode assembly substantially as described above with respect to block 780.

After the polymer coating has been applied, the electrode assemblies may be installed in sensor devices. For example, the electrode assemblies may be physically coupled to a respective PCB or other substrate and electrically coupled to one or more electrical contacts on the PCB or substrate to electrically couple the respective electrode assembly to sensor electronics within the respective sensor device.

Further, it should be appreciated that one or more blocks of the method 800 is optional. For example, blocks 830 and 840 may be omitted in examples where an electrode assembly includes only one electrode. Further block 860 may be omitted if laser cutting is not desired or otherwise is not available. Block 890 may be omitted if a polymer coating is not desirable or available. Further, the orderings of the blocks shown in FIG. 8 may be changed according to various examples according to particular implementations.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

That which is claimed is:

1. A wearable biosensor device comprising:
a housing attachable to a wearer's skin;
an electrode assembly holder disposed within the housing, the electrode assembly holder comprising:
a printed circuit board (“PCB”); and
a plurality of electrical contacts formed on the PCB; and
an electrode assembly physically coupled to the PCB, the electrode assembly having a planar surface and having an invasive end and a device end, the electrode assembly comprising:
a stack of alternating insulating and electrode layers, each layer screen-printed on a previous layer, and the stack formed on a substrate layer;
a first chemical sensing material disposed on an invasive end of a first electrode layer of the electrode layers, a second chemical sensing material disposed on an invasive end of a second electrode layer of the electrode layers, the second chemical sensing material different from the first chemical sensing material;
a polymer coating covering at least a portion of the invasive end of the electrode assembly; and
wherein:
the invasive end of the electrode assembly is insertable beneath the wearer's skin to sense multiple different analyte materials in the wearer's interstitial fluid;
a device end of each electrode of the electrode assembly is electrically coupled to one of the electrical contacts; and
the housing is configured to enable the invasive end of the electrode assembly to extend outside of the housing.

2. The wearable biosensor device of claim 1, wherein the electrode assembly is coupled to the electrode assembly holder and is disposed within the housing.

3. The wearable biosensor device of claim 1, wherein the electrode assembly is coupled to the electrode assembly holder and is disposed at least partially within the housing.

4. The wearable biosensor device of claim 1, wherein the electrode assembly is configured to couple to the plurality of electrical contacts of the electrode assembly holder.

5. The wearable biosensor device of claim 1, further comprising at least one of a counter electrode or a reference electrode, wherein the counter electrode or the reference electrode comprises a non-invasive conductive pad external to the housing.

6. The wearable biosensor device of claim 1, wherein the first and second sensing chemical materials each comprise one of (i) glucose oxidase, (ii) an alcohol oxidase, (iii) a cholesterol oxidase, or (iv) lactate oxidase.

7. The wearable biosensor device of claim 1, wherein the analyte materials comprise two or more of glucose, an alcohol, a cholesterol, or lactate.

8. The wearable biosensor device of claim 1, wherein the polymer coating provides biocompatibility during the wearing of the sensor by the wearer.

9. A method comprising:
providing a substrate material, the substrate material comprising a non-conductive material;
screen-printing an electrode on the substrate material using a conductive material, the electrode having first and second ends opposite each other;
applying a chemical sensing material to the first end of the electrode; and
applying a polymer coating to at least an invasive end of the electrode.

10. The method of claim 8, wherein:
screen-printing the electrode comprises screen-printing a plurality of electrodes on the substrate material;
applying the chemical sensing material to the first end of the electrode comprises applying the chemical sensing material to the first end of at least one electrode of the plurality of electrodes; and
separating the electrode from the substrate material comprises separating each electrode of the plurality of electrodes from the substrate material; and
further comprising:
singulating each electrode of the plurality of electrodes from the other electrodes.

11. The method of claim 8, further comprising:
screen-printing an insulation layer on the electrode using a dielectric ink, the insulation layer not covering at least a portion of at least one end of the electrode; and
screen-printing a second electrode on the insulation layer, the second electrode not covering a portion of at least one end of the insulation layer.

12. The method of claim 8, wherein the electrode comprises a platinized carbon ink or a carbon ink.

13. The method of claim 8, wherein the electrode is a first electrode layer, and further comprising forming a stack of alternating insulating and electrode layers on the first electrode layer by iteratively:
screen-printing an additional insulation layer on a preceding electrode layer using a dielectric ink, the insulation layer not covering at least a portion of at least one end of the preceding electrode layer; and
screen-printing an additional electrode layer on the preceding additional insulation layer; and
wherein applying the polymer coating to the electrode comprises applying the polymer coating to at least an invasive portion of the formed stack of alternating insulating and electrode layers.

14. The method of claim 12, further comprising applying an additional chemical sensing material to at least one of the additional electrode layers.

15. The method of claim 8, further comprising laser-cutting the electrode.

16. The method of claim 8, wherein the chemical sensing material comprises an oxidase enzyme.

17. The method of claim 15, wherein the oxidase enzyme comprises glucose oxidase, an alcohol oxidase, a cholesterol oxidase, lactate oxidase, or any combination thereof.

18. An electrode assembly comprising:
a screen-printed electrode having first and second ends;
a chemical sensing material disposed on the first end of the electrode; and
a polymer coating covering at least a portion of an invasive end of the screen-printed electrode and the chemical sensing material.

19. The electrode assembly of claim 17, further comprising a stack of alternating screen-printed insulating and screen-printed electrode layers, each screen-printed electrode layer separated from an adjacent screen-printed electrode layer by a screen-printed insulation layer, and wherein:
the screen-printed electrode is one of the screen-printed electrode layers, and
each screen-printed electrode layer has a first end, and wherein the first end of each screen-printed electrode layer is covered by neither (i) a screen-printed insulation layer, nor (ii) another screen-printed electrode layer.

20. The electrode assembly of claim 17, wherein the electrode assembly does not include either or both of a counter electrode or a reference electrode.

21. The electrode assembly of claim 17, wherein the screen-printed electrode comprises a platinized carbon ink, or a carbon ink

22. A wearable biosensor device comprising:
a housing attachable to a wearer's skin;
a electrode assembly holder disposed within the housing, the electrode assembly holder comprising:
a substrate;
a plurality of electrical contacts formed on the substrate; and
an electrode assembly electrically coupled to the plurality of electrical contacts, the electrode assembly having a planar surface and comprising:
a first screen-printed electrode having first and second ends;
a chemical sensing material disposed on the first end of the electrode; and
a polymer coating applied to the electrode; and
wherein the first end of the screen-printed electrode extends outside of the housing and wherein a second end of each electrode is electrically coupled to one electrical contact of the plurality of electrical contacts.

23. The wearable biosensor device of claim 21, wherein the first screen-printed electrode comprises a platinized carbon ink or a carbon ink

24. The wearable biosensor device of claim 21, wherein the electrode assembly further comprises a stack of alternating screen-printed insulating and screen-printed electrode layers, each screen-printed electrode layer separated from an adjacent screen-printed electrode layer by a screen-printed insulation layer, and wherein:
the first screen-printed electrode is one of the screen-printed electrode layers, and
each screen-printed electrode layer has a first end extending outside of the housing, and wherein the first end of each screen-printed electrode layer is covered by neither (i) a screen-printed insulation layer, nor (ii) another screen-printed electrode layer.

25. The wearable biosensor device of claim 21, wherein the chemical sensing material comprises an oxidase enzyme.

26. The wearable biosensor device of claim 24, wherein the oxidase enzyme comprises glucose oxidase, an alcohol oxidase, a cholesterol oxidase, or lactate oxidase.

27. The wearable biosensor device of claim 21, wherein the wearable biosensor device comprises a continuous glucose monitor.
